(19) United States
(12) Patent Application Publication
(10) Pub. No.: US 2024/0361169 A1
(43) Pub. Date: Oct. 31, 2024

(54) HYDRATION MONITORS AND SYSTEMS

(71) Applicant: SMART HYDRATION LIMITED, Buckinghamshire (GB)

(72) Inventors: Sean REEL, Buckinghamshire (GB); Andrew STACEY, Buckinghamshire (GB); Anatoli KRASSAVINE, Banbury Oxfordshire (GB); Richard HARLOW, Wales (GB)

(21) Appl. No.: 18/566,379

(22) PCT Filed: Jun. 1, 2021
(86) PCT No.: PCT/GB2021/051351

§ 371 Date: Dec. 1, 2023

(51) Int. Cl. G01F 23/20; A47G 19/22

(52) U.S. Cl. CPC G01F 23/20; A47G 2019/2238

(57) ABSTRACT

A hydration monitor comprising: a container (1) for holding and dispensing a hydration liquid: a volume sensor operable to derive data dependent on the current quantity of the hydration liquid in the container: a location sensor operable to derive data dependent on the current location of the container.

This disclosure relates to hydration monitors and systems for monitoring and/or managing hydration including one or more such monitors. The disclosure may be applicable to use in healthcare, wellness and other contexts.

Hydration is critical to health and becomes even more critical during illness.

In healthcare settings, e.g. hospitals or care homes, it can be time consuming and onerous to ensure that patients remain properly hydrated. To remain properly hydrated, patients need to drink regularly. For instance, a jug of water may be left on a bedside table for a patient to fill a cup whenever the patient wants to have a drink or is prompted to have a drink. To ensure that patients remain properly hydrated, healthcare staff may have to spend significant amounts of time monitoring patients' fluid intake and prompting patients to take on enough fluid. Healthcare staff often may not have sufficient time and resources to ensure that patients remain properly hydrated.

If a patient does not remain properly hydrated, then the patient can fall ill. To recover, the patient may require specialist and lengthy treatment, which may require a period of hospitalisation. On occasion, such an illness might lead to undesirable complications with a patient's other health problems.

GB2575349A discloses a patient hydration monitor providing a visual indication of compliance.

It would be beneficial to mitigate or at least reduce one or more of the problems associated with the prior art.

A first aspect provides a hydration monitor comprising: a container for holding and dispensing a hydration liquid: a volume sensor operable to derive data dependent on the current quantity of the hydration liquid in the container: a location sensor operable to derive data dependent on the current location of the container.

The hydration monitor may comprise a memory for storing the data representative of the current quantity of hydration liquid in the container and the data representative of the current location of the container, the data stored being updated according to change with time in the data derived by the volume sensor and the location sensor.

The hydration monitor is able to derive and, optionally, store data representative of the current quantity of hydration liquid in the container and the current location of the container. This combination of data may provide for more accurate hydration monitoring and management for a given person within an area to be monitored, since tracking the location of the container may help to identify and discount occasions when the hydration fluid has been dispensed from the container, but not drunk, e.g. used to water flowers or poured down a sink or toilet.

The volume sensor may be operable to derive data representative of the current quantity of hydration liquid in the container in accordance with reflections from a surface of the hydration liquid, e.g. a top-surface or an under-surface, received by a receiver of a light beam or an audio beam transmitted from a transmitter. The light beam or the audio beam may be pulsed or continuous.

The volume sensor may be located beneath, or at the bottom of, the container.

In one implementation, the volume sensor may include a transmitter such as a piezoelectric transmitter for transmitting a beam of ultrasonic pulses through the hydration liquid for downward reflection from an under-surface of the hydration liquid to a receiver. The functions of the transmitter and the receiver may be shared in a single device, e.g. a single piezoelectric device, or provided by separate devices.

In another implementation, the volume sensor may be operable to derive data dependent on the current quantity of hydration liquid in the container from a change in the combined weight of the container and the hydration liquid therein, resulting from a change in the amount of hydration liquid in the container. A measure of weight may be derived electronically by one or more force-sensors located beneath, or at the bottom of, the container.

The volume sensor may be located above or at the top of the container. In such an implementation, the volume sensor may comprise a transmitter arranged to transmit a light beam on to a top-surface of the hydration liquid for upward reflection towards a receiver arrange to receive a light beam reflected by the top-surface of the hydration liquid.

The location sensor may comprise a first part on or in the container and a second part at a relatively fixed location relative to the container. The first part and the second part may be in wireless communication with each other, e.g. via infrared, Bluetooth® or WiFi. Data dependent on the distance of the first part from the second part may be obtainable from the wireless communication between the first part and the second part. Data dependent on the current location of the container may be derivable from the data dependent on the distance of the first part from the second part.

The hydration monitor may comprise an orientation sensor operable to derive data dependent on the current orientation of the container. The memory may store the data representative of the current orientation of the container. The data stored may be updated according to change with time in the data derived by the orientation sensor.

For instance, the orientation sensor may comprise an accelerometer.

By deriving and storing data representative of the current orientation of the container, it may be possible to identify instances when the hydration liquid has been dispensed from the container, e.g. when a person may have had a drink. In addition, it may be possible to identify instances when the container has been accidentally knocked over or fallen off an item of furniture such as a bedside table.

The hydration monitor may comprise a temperature sensor operable to derive data dependent on the current temperature of the hydration liquid in the container. The memory may store the data representative of the current temperature of the hydration liquid in the container. The data stored may be updated according to change with time in the data derived by the temperature sensor.

The temperature sensor may comprise a thermistor. In other implementations, the temperature sensor may comprise a thermocouple, a resistance temperature detector or a semiconductor-based sensor.

Advantageously, the temperature sensor may provide a safety means that can be used to determine if the temperature of the hydration liquid in the container is safe for human consumption. Similarly, data derived from measurements made by the temperature sensor may be used to, for example, determine the preferred drinking temperature of a given person to whom the hydration monitor has been assigned. The temperature senor may also be used to determine when a heated hydration fluid, e.g. a hot drink, is safe for consumption.

The container may have any suitable form and may be of any capacity. For example, the container may have the form of a jug, a bottle, a carafe, a cup, a mug or a beaker.

The container may comprise a base and a continuous side wall extending upwards from the base. The continuous side wall may surround an internal volume of the container.

The hydration monitor may comprise a combination of a container and a hub. The container and the hub may be interengagable with one another. In some embodiments, the hub may constitute a smart hub.

The hub may be connectable or connected to the base of the container. The hub may be connected detachably or permanently to the base of the vessel. The hub may be connected to the container by any suitable detachable or permanent connection means.

The hub and the container may be detachably connectable using a magnetic means. The container and/or the hub may comprise one or more magnets allowing the container and the hub to connect and maintain a connection. In some embodiments, both the hub and the container may comprise one or more magnets. In some embodiments, the hub or the container may comprise one or more magnets and the other of the hub or the container may comprises a ferrous material. The magnet(s) and/or the ferrous material may be in the form of a plate, a plurality of plates, modules, discs or the like to enable a magnetic connection.

In some embodiments, the hub and the container may be configured to be connected by a mechanical connection means such as a bayonet connection, screw threads, spring locks, snap on and off fixtures, sliding or rail fixtures or pressure/friction fixtures. In some embodiments, the hub and the container may be permanently connected to each other. In other words, the hydration monitor may not comprise a separate hub and container.

The hydration monitor may comprise a lid arrangable at least partially over the internal volume of the container. The lid may be detachably connected to the container. The lid may be connected to the container by a moveable connection means such as a pivot joint, or the like. The lid may be configured to prevent or reduce the risk of spilling the hydration liquid held within the container. In some embodiments, when the lid is in place the internal volume of the container may be substantially sealed.

The container may comprise at least one handle. In some embodiments, the container may comprise a single handle connected to the container in any suitable way. For example, the container may be connected to the or a continuous side wall of the container. In some embodiments, the container may comprise two handles at diametrically opposed locations, for example. The one or more handles may be configured to allow a user to more easily pick up the container to move it, drink from it, or pour hydration fluid into another vessel or the like. The provision of one or more handles may be useful for older or infirm users or those with joint or muscle conditions to improve their grip on the container.

Each handle may comprise, or be made of, one or more brightly coloured materials to aid visibility of the handle. The handle(s) may be made from the same material as the container. The handle may be connected to the container by any suitable means. The handle may be moulded as a unitary component of the container, moulded or connected to the container using an additional process, connected to the container using a mechanical means, or bonded to the container using an adhesive, for example.

The hydration monitor may comprise a hydration sensor operable to derive data dependent on the current hydration of a person in close proximity to the hydration monitor. The inclusion of a hydration sensor may provide additional useful data for monitoring and/or managing the hydration of a person to whom the hydration sensor is assigned, in use.

The hydration sensor may be operable to derive data dependent on the current hydration of a person when the person touches the hydration sensor.

The hydration sensor may comprise, for example a bio-impedance sensors or a galvanic skin resistance sensor.

Alternatively or additionally, the hydration sensor may comprise a light sensor and/or a photographic sensor.

The light sensor and/or the photographic sensor may be operable to record an image of the person's skin. From the image of the person's skin it may be possible to discern, for example, the person's skin pigmentation and structure. Consequently, data representative of the person's current hydration may be derived from the image.

In an example implementation, the light sensor and/or the photographic sensor may comprise an optical spectrometer.

The hydration sensor may be disposed on or in the container or a part thereof.

In an example implementation, one or more hydration sensors may be disposed on or in one or more handles of the container.

One or more hydration sensors may be disposed in or on the or a lid or a portion thereof.

One or more hydration sensors may be disposed in or on the or a hub or a portion thereof.

In embodiments comprising a lid, the hydration monitor may comprise a sensor operable to derive data dependent on the current location and/or orientation of the lid.

The hydration monitor may comprise one or more magnets and one or more magnet sensors. One or more of the magnet sensors may comprise a magnetometer. The one or more magnets and magnet sensors may be disposed at any suitable location. For example, one or more magnets may be disposed within the or a lid and one or more magnet sensors may be disposed near to the or a lid such that the magnet sensors are able to detect the magnetic field of the magnets. The magnet sensors may be operable to detect relative movement and/or orientation between the magnet(s) and the magnet sensor(s). In this way, the magnet sensor may be operable to detect when the or a lid has been opened and/or removed from the container.

One or more magnets may be disposed within the hub and one or more magnet sensors may be disposed in the container, or vice versa, such that the magnet sensors are able to detect the magnetic field of the magnets. The magnet sensors may be operable to detect relative movement and/or orientation between the magnet(s) and the magnet sensor(s). In this way, the magnet sensor may be operable to detect when the hub has been removed from the container.

The hydration monitor may comprise at least one integrated circuit.

The hydration monitor may comprise a local memory to store data recorded by the sensors.

The hydration monitor may comprise a data transfer device operable to transmit or transfer data stored in the local memory to a data processing device. The hydration monitor may comprise any suitable means for enabling a wired or wireless data transmission or transfer to and/or from the data processing device. The data processing device may be located remotely from the hydration monitor.

The hydration monitor may comprise any suitable means for enabling wireless data transmission to and/or from the or a data processing device. The hydration monitor may be operable to receive and/or transmit data via a Bluetooth connection. The hydration monitor may be operable to receive and/or transmit data via one or more infrared transmitter and reader.

The hydration monitor may be operable to connect to a Wi-Fi network. The hydration monitor may be operable to receive and/or transmit data via a Wi-Fi connection.

Advantageously, the locally stored data being transferred or transmitted to a data processing device may enable the data to be processed, analysed and/or augmented at a location remote from the device. In this way, a user may be able to review data from the hydration monitor, or a plurality of hydration monitors, from a location remote from the hydration monitor(s) such as from another room in the same building, for example.

The hydration monitor may comprise a power source such as a battery or the like. The container may comprise a power source such as a battery or the like. In embodiments comprising a hub, the hub may comprise a power sources such as a battery or the like. Alternatively, the hub may be configured to be connectable to a mains electricity supply. The or each battery may comprise any suitable type of battery.

The or each battery may be rechargeable. The hydration monitor may comprise a wired or wireless battery charging means. For instance, the hub and/or the container may comprise an induction coil configured to charge the battery or batteries.

In some embodiments, the hydration monitor may comprise a socketed power connection configured for charging using a removable cable. In some embodiments the hydration monitor may be configured to be connected to an external power supply such as a mains electricity supply through a wall socket or other power supply such as an external battery or the like.

The hydration monitor may comprise one or more light sources such as one or more light emitting diodes (LEDs). The light source(s) may be configured to emit one or more colours of light. The light source(s) may be configured to emit different colours of light corresponding to one or more indicators, such as hydration monitor error, connecting to a network, connected to a network, user alerts, time period reminders or refill reminders, for example.

The one or more light sources may comprise a plurality of colours and may comprise one or more colour changing LEDs and/or an array of LEDs. Each light source present may comprise a plurality of discrete light source elements. The light source(s) may be operable to create one or more patterns. Each light source may be operable to be controlled individually and separately to any other light sources. Any one or more light source may comprise an LED screen or an electronic ink screen, for example.

The light source(s) may be disposed at any suitable location on the hydration monitor. For instance, one or more light sources may be disposed at or near the container, the or a hub and/or the or a lid, for example.

Advantageously, the one or more light sources may assist a user with visibility. The one or more light sources may assist a user in low light levels such as at night. The one or more light sources may assist a user in identifying different portions of the device in situations of low light levels and/or may assist a user's visibility of other objects within the surrounding space. Additionally, the one or more light sources may assist a user with a visual impairment.

In use, data from the sensors may be transferred to the memory at regular or irregular intervals. Data may be transferred to the memory at pre-determined time intervals. Data may be transferred to the memory upon a sensor detecting a change such as a volume sensor detecting a change in volume of fluid held in the container, for example.

In use, data from the sensors may be transferred to a data processing device via the means suitable for enabling data transmission. The data may be transferred to a data processing device at regular intervals, such as a pre-determined time interval for example. The data may be transferred to a data processing device upon a sensor detecting a change such as a volume sensor detecting a change in volume of hydration liquid held in the container, for example.

In some embodiments, data may not be stored locally in the hydration monitor. In such an embodiment, data from the sensors may be transferred continuously to a further device comprising a memory suitable for storing data from the device. The hydration monitor may be configured to store data corresponding to a pre-determined target. The pre-determined target may relate to the consumption of hydration liquid. The pre-determined target may relate to the consumption of hydration liquid by a user over a pre-determined time period. The pre-determined target may be set by a user. Data relating to the pre-determined target may be stored on the or a memory associated with the hydration monitor. The pre-determined time period may be 6 hours, 12 hours, 24 hours or 48 hours, for example.

The hydration monitor may be operable to indicate to a user whether the given person has consumed a volume of hydration liquid that is below the target, near to or at the target, or above the target, for example.

The hydration monitor may be operable to indicate such information to a user via one or more of a visual and/or audio signal.

The visual signal may be provided by the one or more light sources. For example, a different pattern or colour of light may be presented corresponding to the given person's consumption of fluid in relation to the pre-determined target.

The audio signal may be produced by a speaker housed on or in the hydration monitor. For instance, the speaker may be housed on or in the container or housed in the or a hub, e.g. the or a smart hub. Alternatively or additionally, the audio signal may be relayed through an audio device connected to the hydration monitor via a cable or a wireless connection, e.g. Bluetooth, infrared of WiFi.

In this way, the given person may be provided with an automatic indication as to whether their fluid consumption is too low or adequate. This may be particularly beneficial for people who have difficulties with memory. This may also be particularly beneficial to other users such a medical staff or care workers, as they may also be provided with an automatic and immediate indication of a given person's hydration liquid consumption without the need for any calculations.

A second aspect provides a system for monitoring hydration of one or more people within an area to be monitored comprising:
one or more hydration monitors, each hydration monitor being assigned to a given person within the area to be monitored;
a data processing device arranged to:
   receive raw data from the one or more hydration monitors; and
   process the raw data to produce one or more final reportable events;
a database arranged to store data relating to the or each final reportable event; and
a presentation engine operably connected to the database and arranged to produce a presentation utilising the data relating to one or more of the final reportable events.

It will be appreciated that the system monitors hydration of one or more given persons within an area to be monitored and provides a presentation to facilitate hydration management for the given person(s) to whom the hydration monitors are assigned.

One or more of the hydration monitors may be a hydration monitor according to the first aspect.

By making use of raw data relating to more than just the volume of hydration liquid in the container, e.g. raw data relating to at least the volume of hydration liquid in the container and the location of the container, better monitoring and/or monitoring of hydration may be facilitated. By using these two or more sources of raw data, it has been found that a better, more accurate picture of hydration may be created. In contrast, systems which collect as raw data only data relating to the volume of hydration liquid in the container provide a picture of how and when hydration liquid is poured from or added to the container. However, this picture may not correspond with hydration. By using the hydration monitors and systems according to the present disclosure, it is possible to be more confident that the data provide an accurate picture of a person's hydration.

The presentation may be a visual presentation. The presentation may be an audio presentation. The presentation may be a multimedia presentation.

The hydration monitor may continuously monitor the volume of hydration liquid in the container and the location of the container.

The hydration monitor may also monitor one or more of: the temperature of the hydration liquid in the container; the orientation of the container; and the given person's hydration level.

Raw data from the sensors in the hydration monitor may be stored locally in a memory associated with the hydration monitor.

The data processing device may be termed an analytics engine. The data processing device may comprise an analytics engine memory and a processor.

The raw data may be transferred or transmitted continuously or at intervals. For instance, the raw data may be transferred or transmitted to the data processing device at regular intervals, e.g. at scheduled intervals and/or in response to certain events. For instance, the raw data may be transferred or transmitted to the data processing device whenever a change is detected by one or more of the sensors of the hydration monitor. For instance, the sensors may detect changes in the volume of hydration liquid in the container, changes in the location of the container and/or changes in the orientation of the container.

The raw data may be transferred or transmitted to the data processing device wirelessly or by a cable.

The data processing device may be located remotely from the one or more hydration monitors.

The data processing device may employ statistical and/or artificial intelligence (AI) and/or machine learning techniques in carrying out its functions.

The raw data received from the hydration monitor(s) may be stored in the analytics engine memory.

The processor may process the raw data to identify one or more events of potential interest. The or each event of potential interest may be assigned a confidence level, which may be compared with a confidence threshold.

The processor may process the raw data to identify one or more reportable events. The or each reportable event may be assigned a confidence level, which may be compared with a confidence threshold.

In the data processing device, the processor may be arranged to carry out a processing method including one or more of the following steps.

In a first step of the processing method, the processor may pre-process the raw data, including validating and cleaning the raw data, to produce clean data. The clean data may be stored in the analytics engine memory.

In a subsequent step of the processing method, the processor may analyse the clean data to identify potential events of interest. Events of interest may include, for example, occasions when there was a change in the amount of fluid in the container of a hydration monitor assigned to a given person. For example, a change in the amount of fluid in a given container may be indicative of the given person having had a drink. In another example, a combination of readings corresponding to a sudden movement of the container, a decrease in the amount of hydration liquid in the container and an extended period of time in an extreme position may be indicative of the dropping of the container, which may, particularly in a healthcare setting, require that a care worker goes to the assistance of the given person. Such a combination of readings may also potentially indicate an accident, e.g. a fall, for the given person. Again, this may mean that the given person requires the assistance of a care worker.

The processor may then assign a confidence level to each potential event of interest. The confidence level may be checked and revised on a continuous basis, a quasi-continuous basis or periodically, as new data are supplied to the data processing device or analytics engine.

If the confidence level does not pass a first confidence threshold, then the potential event of interest may be considered erroneous or should be consolidated. In this case, the potential event of interest may be labelled a non-event, in a step of the processing method.

In another step of the processing method, an event of potential interest may become a reportable event if the confidence level assigned to the event of potential interest passes the first confidence threshold.

In a subsequent step of the processing method, the confidence level assigned to the or each reportable event may be assessed against a second confidence threshold. If the confidence level of the reportable event passes the second confidence threshold, then the reportable event may be marked as a final reportable event. The data processing device or analytics engine may be arranged such that it cannot update or revise a given reportable event once it has been marked as a final reportable event.

If the confidence level of the reportable event does not pass the second confidence threshold, then the reportable event may be considered erroneous or should be consolidated. In this case, the reportable event may be labelled a non-event.

The step of labelling an event as a non-event may be applied to potential events of interest with a confidence level that does not pass the first confidence threshold or to reportable events with a confidence level that does not pass the second confidence threshold.

The presentation engine may be arranged to augment the presentation with data from one or more other sources, including, but not limited to, a user identity profile associated with a given person for a given hydration monitor and/or a hydration profile associated with a given person for a given hydration monitor.

The hydration profile may be based on past measurements and analysis of a given person's levels of hydration and/or may be benchmarked or otherwise take into account statistical, population-level data, e.g. relating to desirable or typical hydration profiles for other comparable members of the wider population.

By augmenting the presentation with data from one or more other sources, the presentation engine may produce an enriched report, which may be bespoke for a given person.

Conveniently, the presentation, e.g. the or a bespoke report, produced by the presentation engine may be displayed, on a monitor or other display device, e.g. via a web portal or application.

The presentation, e.g. the or a bespoke report, may be displayed on a mobile phone screen, a laptop screen, a desktop computer monitor, a tablet computer or the like, for example.

Accordingly, the system may facilitate hydration monitoring and/or management for a given person. Consequently, the likelihood of the given person falling ill due to not being properly hydrated may be reduced. Accordingly, the complications, time and expense of treating a person who has fallen ill due to not being properly hydrated may be minimised or even eliminated.

The system may comprise any number of hydration monitors. The hydration monitors may be assigned to different people and/or different locations within the area to be monitored. The area to be monitored may comprise one or more parts of one or more buildings.

In an example implementation, the system may be installed in a care home setting with a hydration monitor assigned to each care home resident. In another example implementation, the system may be installed in a hospital setting with a hydration monitor assigned to each hospital patient.

Via any suitable user interface or input device, a user may be able to access, view and/or manipulate the presentation, or request a further presentation.

A third aspect provides a method for monitoring hydration of one or more people within an area to be monitored comprising:
collecting raw data from one or more hydration monitors, each hydration monitor being assigned to a given person within the area to be monitored;
processing the raw data to produce one or more final reportable events; and
producing a presentation utilising the data relating to one or more of the final reportable events.

One or more of the hydration monitors may be a hydration monitor according to the first aspect.

The method may be performed using a system according to the second aspect.

The skilled person will appreciate that except where mutually exclusive, a feature or parameter described in relation to any one of the above aspects may be applied to any other aspect. Furthermore, except where mutually exclusive, any feature or parameter described herein may be applied to any aspect and/or combined with any other feature or parameter described herein.

Example embodiments will now be described with reference to the accompanying drawings, in which:

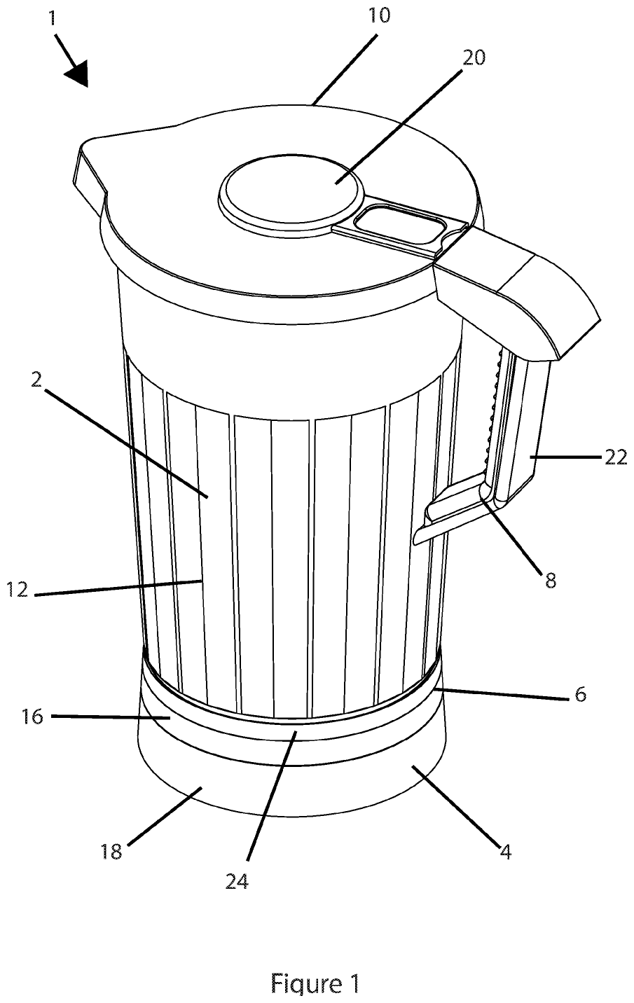

FIG. 1 is a perspective view of a hydration monitor comprising a combination of a jug and a smart hub;

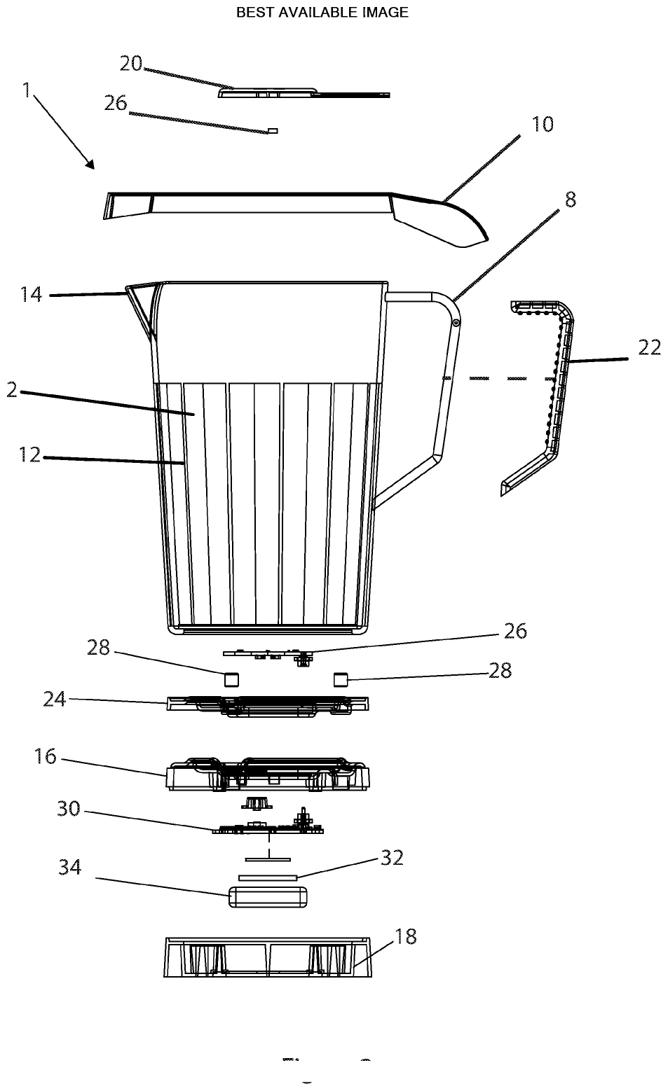

FIG. 2 is an exploded view of the hydration monitor shown in FIG. 1;

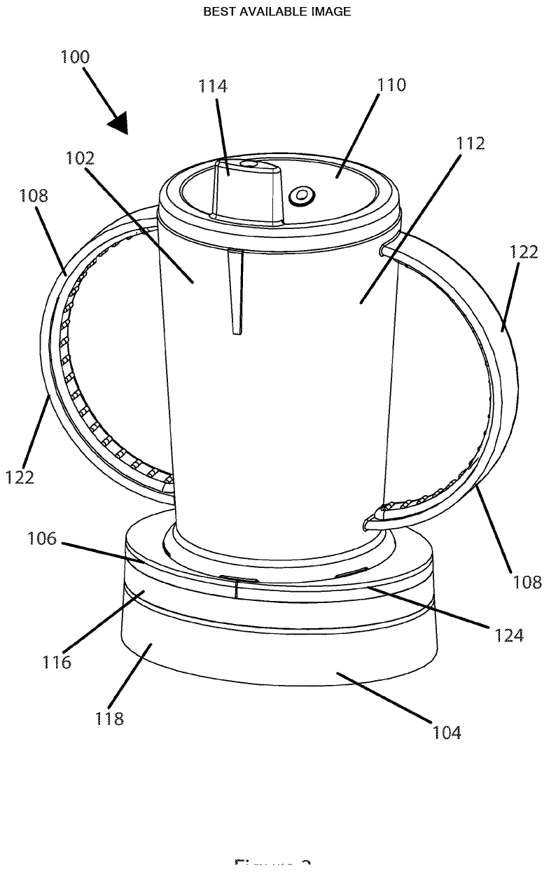

FIG. 3 is a perspective view of a hydration monitor comprising a combination of a beaker and a smart hub;

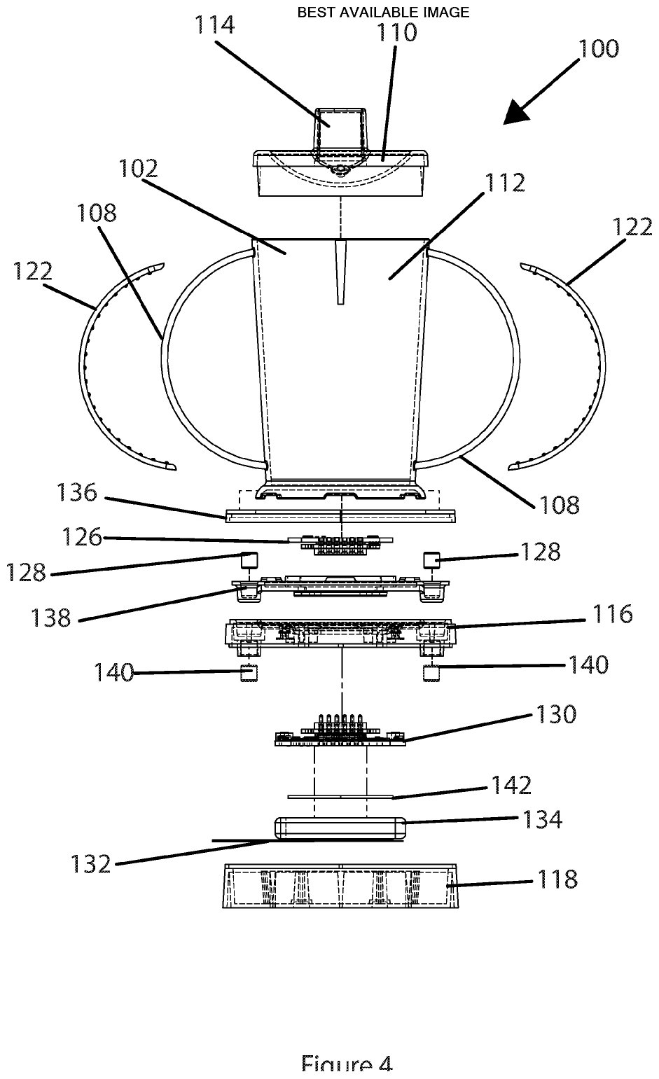

FIG. 4 is an exploded view of the hydration monitor shown in FIG. 3;

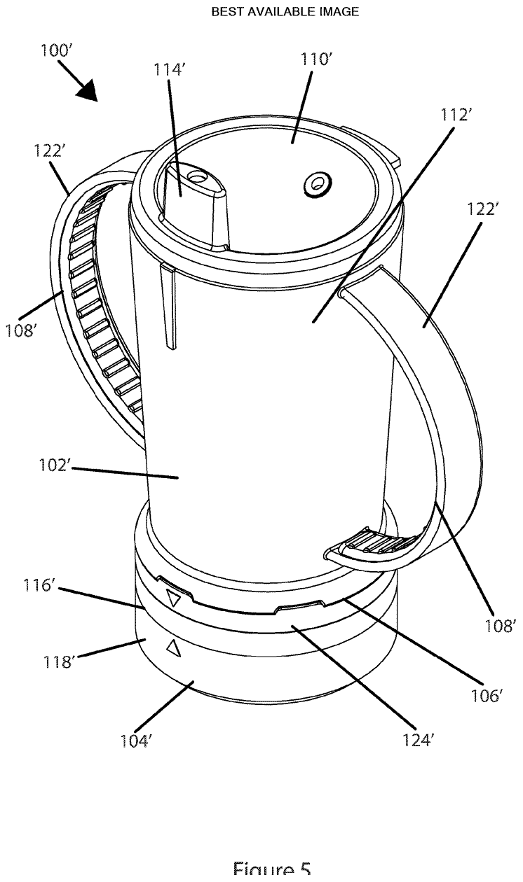

FIG. 5 is a perspective view of another hydration monitor comprising a combination of a beaker and a smart hub;

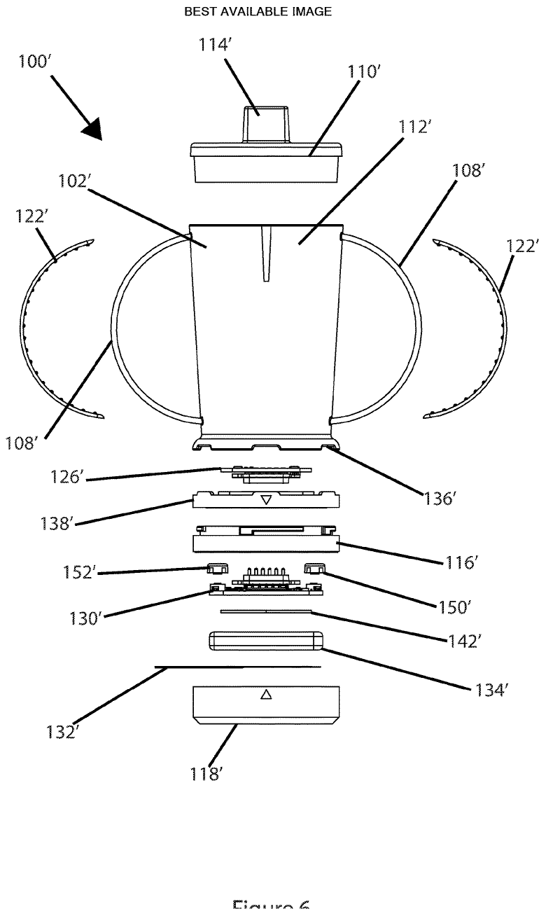

FIG. 6 is an exploded view of the hydration monitor shown in FIG. 5; and

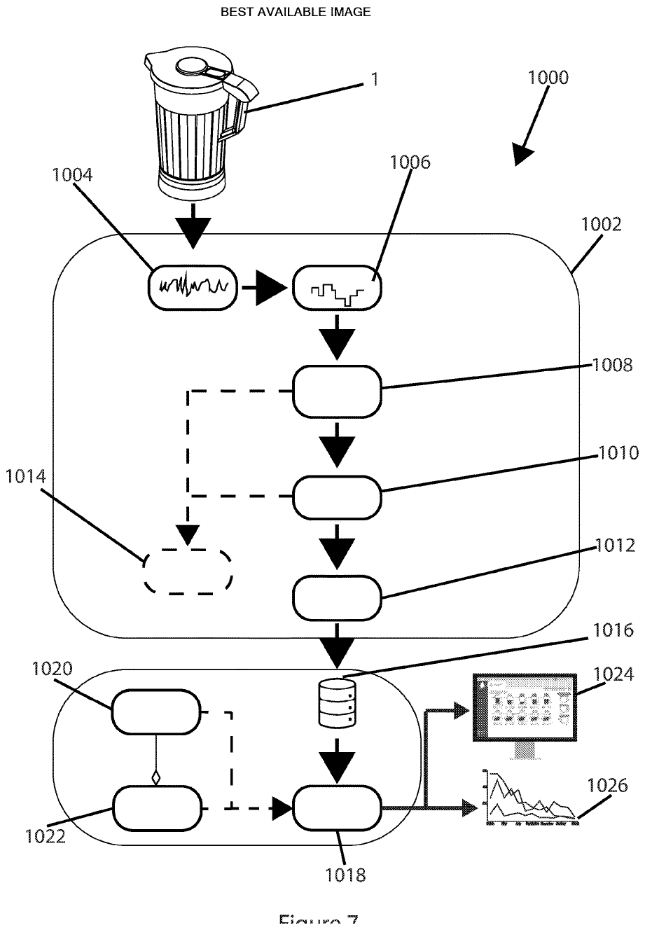

FIG. 7 illustrates schematically a system for monitoring hydration.

FIGS. 1 and 2 show a hydration monitor 1 comprising a combination of a container in the form of a jug 2 and a smart hub 4 connected to an underside of a base portion 6 of the jug 2.

The jug 2 may have any capacity. The jug 2 may have a capacity of at least 200 ml or at least 250 ml. The jug may have a capacity of up to 2 litres. For example, the jug 2 may have a capacity of 200 ml, 250 ml, 500 ml, 750 ml, 1 litre or 1.5 litres.

The jug 2 has a continuous side wall 12 extending upwards from the base portion 6. The continuous side wall 12 extends around an internal volume of the jug 2. A handle 8 extends outwards from the continuous side wall 12. A pouring spout 14 is provided at the top of the continuous side wall 12. The pouring spout 14 is located diametrically opposite from the handle 8. The handle 8 has a softer grip 22 disposed thereon.

A lid 10 sits on top of the continuous side wall 12 and extends over the internal volume of the jug 2, the pouring spout 14 and an upper portion of the handle 8. The lid 10 has a magnet hub 20 attached thereto. The magnet hub 20 has two magnets 26 attached thereto.

The base portion 6 comprises a base shell 24 beneath and sealed from the internal volume of the jug 2. The base shell 24 houses a container circuit board 26 and three magnets 28 equally spaced around an imaginary circle lying in a substantially horizontal plane within the base shell 24. An underside of the base shell 24 is adapted to be connected to the smart hub 4, in use. The underside of the base shell 24 may be termed a connection plate.

The smart hub 4 is disposed beneath the jug 2. In use, the jug 2 may be placed on, and connected to, the smart hub 4. When a user wants to pour liquid from the jug 2 or fill the jug 2 with water from a tap, the user can disconnect or undock the jug 2 from the smart hub 4. Between uses of the jug 2, the jug 2 may be connected to or docked with the smart hub 4.

The smart hub 4 comprises a smart hub top shell part 16 and a smart hub bottom shell part 18. Together, the smart hub top shell part 16 and the smart hub bottom shell part 18 define a cavity. A battery 34, an induction coil 32 and a smart hub circuit board 30 are housed in the cavity.

An upper surface of the smart hub top shell part 16 is adapted to be connected to the underside of the base shell 24 of the jug 2. In this example, the underside of the base shell 24 of the jug 2 and the upper surface of the smart hub top shell part 16 are adapted to be connected to each other magnetically.

An underside of the smart hub bottom shell part 18 is adapted to be stood on a surface such as a table or other item of furniture. For instance, the underside of the smart hub bottom shell part 18 may be provided with one or more non-slip feet (not shown).

The smart hub 4 may be configured to be connectable to an external power source, e.g. a mains electricity supply.

The jug 2 comprises a volume sensor (not shown) operable to derive data dependent on the current quantity of a hydration liquid in the jug 2. The volume sensor includes a transmitter such as a piezoelectric transmitter for transmitting a beam of ultrasonic pulses through the hydration liquid for downward reflection from an under-surface of the hydration liquid to a receiver. The functions of the transmitter and the receiver may be shared in a single device, e.g. a single piezoelectric device, or provided by separate devices, housed in the base portion 6 and operably connected to the container circuit board 26.

The hydration monitor 1 includes a location sensor (not shown) operable to derive data dependent on the current location of the jug 2. The location sensor comprises a first part (not shown) housed in the base portion 6 and operably connected to the container circuit board 26 and a second part (not shown) housed in the smart hub 4 and operably connected to the smart hub circuit board 30. Hence, in use, the location of the second part will generally be relatively fixed relative to the location of the first part. The first part and the second part are in wireless communication with each other, e.g. via infrared, Bluetooth® or WiFi. Data dependent on the distance of the first part from the second part is obtainable from the wireless communication between the first part and the second part. Data dependent on the current location of the container is derivable from the data dependent on the distance of the first part from the second part.

The hydration monitor 1 includes a temperature sensor (not shown) operable to derive data dependent on the current temperature of the hydration liquid in the jug 2. The temperature sensor may comprise a thermistor. In other implementations, the temperature sensor may comprise a thermocouple, a resistance temperature detector or a semiconductor-based sensor.

One or more light emitting diodes (LEDs) are provided in the base portion 6 of the jug 2. The LEDs may provide, in use, a form of night light or lamp. The colours and/or intensities of the emitted light may be variable according to user preference.

The jug 2 comprises an orientation sensor (not shown) operable to derive data dependent on the current orientation of the jug 2. The orientation sensor is housed in the base portion 6 and operably connected to the container circuit board 26. For instance, the orientation sensor may comprise an accelerometer.

The smart hub 4 includes a Wi-Fi and Bluetooth transmitter (not shown) to enable wireless communication with other devices, in particular to transmit raw data from the sensors to a data processing device or analytics engine.

The smart hub 4 comprises one or more indicator lights (not shown), e.g. to provide visual information about the current state of the hydration monitor 1.

A hydration sensor is disposed in the handle 8. The hydration sensor is operable to derive data dependent on the current hydration of a person in close proximity to the hydration monitor. The hydration sensor is operable to derive data dependent on the current hydration of a person when the person touches the hydration sensor. The hydration sensor may comprise, for example a bio-impedance sensors or a galvanic skin resistance sensor.

The hydration monitor 1 comprises a memory for storing locally the data from the sensors. The data stored in the memory is updated according to change with time in the data from the sensors.

The memory may be located in the base portion 6 and/or the smart hub 4.

FIGS. 3 and 4 show a hydration monitor 100 comprising a combination of a container in the form of a beaker 102 and a smart hub 104 connected to an underside of a base portion 106 of the beaker 102.

The beaker 102 may have any capacity.

The beaker 102 has a continuous side wall 112 extending upwards from the base portion 106. The continuous side wall 112 extends around an internal volume of the beaker 102. Two handles 108 extend outwards from diametrically opposed portions of the continuous side wall 112. The handles 108 each have a softer grip 122 disposed thereon.

A lid 110 is fitted to the top of the continuous side wall 112 and extends over the internal volume of the beaker 106. The lid 110 includes a pouring spout 114 through which a user may drink fluid out of the beaker 106.

The base portion 106 comprises a base shell 124 beneath and sealed from the internal volume of the beaker 102. The base shell 124 is formed of an upper base shell part 136 and a lower base shell part 138. The base shell 124 houses a container circuit board 126 and two magnets 128 equally spaced around an imaginary circle lying in a substantially horizontal plane within the base shell 124. An underside of the lower base shell part 138 is adapted to be connected to the smart hub 104, in use. The underside of the lower base shell part 138 may be termed a connection plate.

The smart hub 104 is disposed beneath the beaker 102. In use, the beaker 102 may be placed on, and connected to, the smart hub 104. When a user wants to pour liquid from the beaker 102 or fill the beaker 102 with water from a tap, the user can disconnect or undock the beaker 102 from the smart hub 104. Between uses of the beaker 102, the beaker 102 may be connected to or docked with the smart hub 104.

The smart hub 104 comprises a smart hub top shell part 116 and a smart hub bottom shell part 118. Together, the smart hub top shell part 116 and the smart hub bottom shell part 118 define a cavity. A battery 134, an induction coil 132 and a smart hub circuit board 130 are housed in the cavity. An adhesive pad 142 sticks the battery 134 to the smart hub circuit board 130.

The smart hub 104 includes two magnets 140, which are aligned with the magnets 128 in the base shell 124 when, in use, the beaker 102 is docked with the smart hub 104.

An upper surface of the smart hub top shell part 116 is adapted to be connected to the underside of the lower base shell part 138 of the beaker 102. In this example, the underside of the base shell 124 of the beaker 102 and the upper surface of the smart hub top shell part 116 are adapted to be connected to each other magnetically.

An underside of the smart hub bottom shell part 118 is adapted to be stood on a surface such as a table or other item of furniture. For instance, the underside of the smart hub bottom shell part 118 may be provided with one or more non-slip feet (not shown).

The smart hub 104 may be configured to be connectable to an external power source, e.g. a mains electricity supply.

The beaker 102 comprises a volume sensor (not shown) operable to derive data dependent on the current quantity of a hydration liquid in the beaker 102. The volume sensor includes a transmitter such as a piezoelectric transmitter for transmitting a beam of ultrasonic pulses through the hydration liquid for downward reflection from an under-surface of the hydration liquid to a receiver. The functions of the transmitter and the receiver may be shared in a single device, e.g. a single piezoelectric device, or provided by separate devices, housed in the base portion 106 and operably connected to the container circuit board 126.

The hydration monitor 100 includes a location sensor (not shown) operable to derive data dependent on the current location of the beaker 102. The location sensor comprises a first part (not shown) housed in the base portion 106 and operably connected to the container circuit board 126 and a second part (not shown) housed in the smart hub 104 and operably connected to the smart hub circuit board 130. Hence, in use, the location of the second part will generally be relatively fixed relative to the location of the first part. The first part and the second part are in wireless communication with each other, e.g. via infrared, Bluetooth® or WiFi. Data dependent on the distance of the first part from the second part is obtainable from the wireless communication between the first part and the second part. Data dependent on the current location of the container is derivable from the data dependent on the distance of the first part from the second part.

The hydration monitor 100 includes a temperature sensor (not shown) operable to derive data dependent on the current temperature of the hydration liquid in the beaker 102. The temperature sensor may comprise a thermistor. In other implementations, the temperature sensor may comprise a thermocouple, a resistance temperature detector or a semiconductor-based sensor.

One or more light emitting diodes (LEDs) are provided in the base portion 106 of the beaker 102. The LEDs may provide, in use, a form of night light or lamp. The colours and/or intensities of the emitted light may be variable according to user preference.

The beaker 102 comprises an orientation sensor (not shown) operable to derive data dependent on the current orientation of the beaker 102. The orientation sensor is housed in the base portion 106 and operably connected to the container circuit board 126. For instance, the orientation sensor may comprise an accelerometer.

The smart hub 104 includes a Wi-Fi and Bluetooth transmitter (not shown) to enable wireless communication with other devices, in particular to transmit raw data from the sensors to a data processing device or analytics engine.

The smart hub 104 comprises one or more indicator lights (not shown), e.g. to provide visual information about the current state of the hydration monitor 100.

A hydration sensor is disposed in each of the handles 108. The hydration sensor is operable to derive data dependent on the current hydration of a person in close proximity to the hydration monitor. The hydration sensor is operable to derive data dependent on the current hydration of a person when the person touches the hydration sensor. The hydration sensor may comprise, for example a bio-impedance sensors or a galvanic skin resistance sensor.

The hydration monitor 100 comprises a memory for storing locally the data from the sensors. The data stored in the memory is updated according to change with time in the data from the sensors.

The memory may be located in the base portion 106 and/or the smart hub 104.

FIGS. 5 and 6 show another hydration monitor 100′ comprising a combination of a container in the form of a beaker 102′ and a smart hub 104′ connected to an underside of a base portion 106′ of the beaker 102′.

The beaker 102′ may have any capacity.

The beaker 102′ has a continuous side wall 112′ extending upwards from the base portion 106′. The continuous side wall 112′ extends around an internal volume of the beaker 102′. Two handles 108′ extend outwards from diametrically opposed portions of the continuous side wall 112′. The handles 108′ each have a softer grip 122′ disposed thereon.

A lid 110′ is fitted to the top of the continuous side wall 112′ and extends over the internal volume of the beaker 106′. The lid 110′ includes a pouring spout 114′ through which a user may drink fluid out of the beaker 106′.

The base portion 106′ comprises a base shell 124′ beneath and sealed from the internal volume of the beaker 102′. The base shell 124′ is formed of an upper base shell part 136′ and a lower base shell part 138′. In this example, the upper base shell part 136′ is integrally formed with the beaker 102′. The base shell 124′ houses a container circuit board 126′. An underside of the lower base shell part 138′ is adapted to be connected to the smart hub 104′, in use. The underside of the lower base shell part 138′ may be termed a connection plate.

The smart hub 104′ is disposed beneath the beaker 102′. In use, the beaker 102′ may be placed on, and connected to, the smart hub 104′. When a user wants to pour liquid from the beaker 102′ or fill the beaker 102′ with water from a tap, the user can disconnect or undock the beaker 102′ from the smart hub 104′. Between uses of the beaker 102′, the beaker 102′ may be connected to or docked with the smart hub 104′.

The smart hub 104′ comprises a smart hub top shell part 116′ and a smart hub bottom shell part 118′. Together, the smart hub top shell part 116′ and the smart hub bottom shell part 118′ define a cavity. A battery 134′, an induction coil 132′ and a smart hub circuit board 130′ are housed in the cavity. An adhesive pad 142′ sticks the battery 134′ to the smart hub circuit board 130′.

Disposed upon and connected to the smart hub circuit board 130′ are a reset button 150′ and a standby button 152′. The reset button 150′ and standby button 152′ are disposed at opposing sides of the smart hub circuit board 130′. The reset button 150′ is configured such that when pressed by a user, one or more functions and/or settings of the smart hub circuit board 130′ are reset back to a pre-determined configuration. In some embodiments the reset button 150′ is configured such that it must be held for a pre-determined period of time, such as 5 seconds or 10 seconds for example, before the one or more functions and/or settings are reset. The standby button 152′ is configured such that when pressed by a user, the smart hub circuit board 130′ enters a standby mode. By pressing the standby button 152′ again the smart hub circuit board 130′ will no longer be in the standby mode.

An upper surface of the smart hub top shell part 116′ is adapted to be connected to the underside of the lower base shell part 138′ of the beaker 102′. In this example, the underside of the base shell 124′ of the beaker 102′ and the upper surface of the smart hub top shell part 116′ are adapted to be connected to each other mechanically via a bayonet-type fitting.

An underside of the smart hub bottom shell part 118′ is adapted to be stood on a surface such as a table or other item of furniture. For instance, the underside of the smart hub bottom shell part 118′ may be provided with one or more non-slip feet (not shown).

The smart hub 104′ may be configured to be connectable to an external power source, e.g. a mains electricity supply.

The beaker 102′ comprises a volume sensor (not shown) operable to derive data dependent on the current quantity of a hydration liquid in the beaker 102′. The volume sensor includes a transmitter such as a piezoelectric transmitter for transmitting a beam of ultrasonic pulses through the hydration liquid for downward reflection from an under-surface of the hydration liquid to a receiver. The functions of the transmitter and the receiver may be shared in a single device, e.g. a single piezoelectric device, or provided by separate devices, housed in the base portion 106′ and operably connected to the container circuit board 126′.

The hydration monitor 100′ includes a location sensor (not shown) operable to derive data dependent on the current location of the beaker 102′. The location sensor comprises a first part (not shown) housed in the base portion 106′ and operably connected to the container circuit board 126′ and a second part (not shown) housed in the smart hub 104′ and operably connected to the smart hub circuit board 130′. Hence, in use, the location of the second part will generally be relatively fixed relative to the location of the first part. The first part and the second part are in wireless communication with each other, e.g. via infrared, Bluetooth® or WiFi. Data dependent on the distance of the first part from the second part is obtainable from the wireless communication between the first part and the second part. Data dependent on the current location of the container is derivable from the data dependent on the distance of the first part from the second part.

The hydration monitor 100′ includes a temperature sensor (not shown) operable to derive data dependent on the current temperature of the hydration liquid in the beaker 102′. The temperature sensor may comprise a thermistor. In other implementations, the temperature sensor may comprise a thermocouple, a resistance temperature detector or a semiconductor-based sensor.

One or more light emitting diodes (LEDs) are provided in the base portion 106′ of the beaker 102′. The LEDs may provide, in use, a form of night light or lamp. The colours and/or intensities of the emitted light may be variable according to user preference.

The beaker 102′ comprises an orientation sensor (not shown) operable to derive data dependent on the current orientation of the beaker 102′. The orientation sensor is housed in the base portion 106′ and operably connected to the container circuit board 126′. For instance, the orientation sensor may comprise an accelerometer.

The smart hub 104′ includes a Wi-Fi and Bluetooth transmitter (not shown) to enable wireless communication with other devices, in particular to transmit raw data from the sensors to a data processing device or analytics engine.

The smart hub 104′ comprises one or more indicator lights (not shown), e.g. to provide visual information about the current state of the hydration monitor 100′.

A hydration sensor is disposed in each of the handles 108′. The hydration sensor is operable to derive data dependent on the current hydration of a person in close proximity to the hydration monitor. The hydration sensor is operable to derive data dependent on the current hydration of a person when the person touches the hydration sensor. The hydration sensor may comprise, for example a bio-impedance sensors or a galvanic skin resistance sensor.

The hydration monitor 100′ comprises a memory for storing locally the data from the sensors. The data stored in the memory is updated according to change with time in the data from the sensors.

The memory may be located in the base portion 106′ and/or the smart hub 104′.

FIG. 7 illustrates schematically a system for monitoring hydration. The system 1000 comprises at least one hydration monitor. Each hydration monitor may be one of the hydration monitors disclosed herein, e.g. the hydration monitor 1, the hydration monitor 100 or the hydration monitor 100′. In FIG. 7, the hydration monitor 1 is shown. The hydration monitor 1 is assigned to a given person within an area to be monitored.

The hydration monitor 1 continuously monitors the volume of fluid in the container and the location of the container. The hydration monitor 1 may also monitor the temperature of the fluid in the container, the orientation of the container and the given person's hydration level. Raw data from the sensors in the hydration monitor is stored locally in the memory associated with the hydration monitor 1. The hydration monitor 1 transfers or transmits this raw data to an analytics engine 1002 comprising an analytics engine memory and a processor. The raw data may be transferred or transmitted continuously or at intervals. For instance, the raw data may be transferred or transmitted to the analytics engine 1002 at regular intervals, e.g. at scheduled intervals and/or in response to certain events. For instance, the raw data may be transferred or transmitted to the analytics engine 1002 whenever the container changes location and/or changes orientation.

The raw data may be transferred or transmitted to the analytics engine 1002 wirelessly or by a cable. The analytics engine 1002 may be located remotely from the hydration monitor 1.

In general, the analytics engine 1002 is operable to convert the raw data into usable information for monitoring and/or managing hydration. The analytics engine 1002 may employ statistical and/or artificial intelligence (AI) and/or machine learning techniques in carrying out its functions.

Referring to the example system 1000 illustrated in FIG. 7, the analytics engine 1002 is operable to carry out the following steps.

In a first step 1004, the raw data received from the hydration monitor(s) 1 is stored in the analytics engine memory. The raw data is stored “as is” for audit and compliance reasons.

In the analytics engine 1002, the processor is arranged to carry out a processing method including the following steps:

In a first step 1006 of the processing method, the processor pre-processes the raw data, including validating and cleaning the raw data, to produce clean data. The clean data is stored in the analytics engine memory.

In a subsequent step 1008 of the processing method, the processor analyses the clean data to identify potential events of interest. Events of interest may include, for example, occasions when there was a change in the amount of fluid in the container of a hydration monitor 1 assigned to a given person. A change in the amount of fluid in a given container may be indicative of the given person having had a drink.

The processor then assigns a confidence level to each potential event of interest. The confidence level may be checked and revised on a continuous basis, a quasi-continuous basis or periodically, as new data is supplied to the analytics engine 1002.

If the confidence level does not pass a first confidence threshold, then the potential event of interest is considered erroneous or should be consolidated. In this case, the potential event of interest is labelled a non-event, in a step 1014 of the processing method.

In another step 1010 of the processing method, an event of potential interest becomes a reportable event if the confidence level assigned to the event of potential interest passes the first confidence threshold.

In a subsequent step 1012 of the processing method, the confidence level assigned to the or each reportable event is assessed against a second confidence threshold. If the confidence level of the reportable event passes the second confidence threshold, then the reportable event is marked as a final reportable event. The analytics engine 1002 cannot update or revise a given reportable event once it has been marked as a final reportable event.

If the confidence level of the reportable event does not pass the second confidence threshold, then the reportable event is considered erroneous or should be consolidated. In this case, the reportable event is labelled a non-event, in the step 1014 of the processing method. It will therefore be appreciated that the step 1014 of labelling an event as a non-event is applied to potential events of interest with a confidence level that does not pass the first confidence threshold or to reportable events with a confidence level that does not pass the second confidence threshold.

Data relating to the or each final reportable event is stored in a database 1016. A presentation engine 1018 is operably connected to the database 1016 and is arranged to produce a visual presentation utilising the data relating to the or each final reportable event for the or each hydration monitor 1. The presentation engine 1018 may be arranged to augment the visual presentation with data from one or more other sources, including, but not limited to, a user identity profile 1020 associated with a given person for a given hydration monitor 1 and/or a hydration profile 1022 associated with a given person for a given hydration monitor 1. The hydration profile 1022 may be based on past measurements and analysis of a given person's levels of hydration and/or may be benchmarked or otherwise take into account statistical, population-level data, e.g. relating to desirable or typical hydration profiles for other comparable members of the wider population.

By augmenting the visual presentation with data from one or more other sources, the presentation engine 1018 may, in a subsequent step 1026, produce an enriched report, which may be bespoke for a given person.

Conveniently, the visual presentation, e.g. the or a bespoke report, produced by the presentation engine 1018 may be displayed, in a subsequent step 1024 on a monitor or other display device, e.g. via a web portal or application.

Accordingly, the system 1000 may facilitate hydration monitoring and/or management for a given person. Consequently, the likelihood of the given person falling ill due to not being properly hydrated may be reduced. Accordingly, the complications, time and expense of treating a person who has fallen ill due to not being properly hydrated may be minimised or even eliminated.

The system 1000 may comprise any number of hydration monitors. The hydration monitors may be assigned to different people and/or different locations within the area to be monitored. The area to be monitored may comprise one or more parts of one or more buildings.

In an example implementation, the system 1000 may be installed in a care home setting with a hydration monitor 1 assigned to each care home resident.

Systems according to the present disclosure may be installed in other settings where it is desirable to monitor and/or manage hydration of individuals. Such settings may include healthcare or wellness settings.

It will be appreciated that the disclosure may provide a fluid measurement and tracking system, intended for hydration tracking for personal and health care users.

Typically, the fluid utilised may be water, as water constitutes a good hydration liquid. In some implementations, one or more beverages other than solely water, may be employed as a hydration liquid. The hydration liquid employed at any given time may be selected based on a number of factors, including, for example, medical need and individual preference.

It will be understood that the invention is not limited to the embodiments described above. Various modifications and improvements can be made without departing from the concepts disclosed herein. Except where mutually exclusive, any of the features may be employed separately or in combination with any other features and the disclosure extends to all combinations and sub-combinations of one or more features disclosed herein.

1. A hydration monitor comprising: a container for holding and dispensing a hydration liquid; a volume sensor operable to derive data dependent on the current quantity of the hydration liquid in the container; and a location sensor operable to derive data dependent on the current location of the container.

2. The hydration monitor according to claim 1 comprising a memory for storing the data representative of the current quantity of hydration liquid in the container and the data representative of the current location of the container, the data stored being updated according to change with time in the data derived by the volume sensor and the location sensor.

3. The hydration monitor according to claim 1, wherein the volume sensor is operable to derive data representative of the current quantity of hydration liquid in the container in accordance with reflections from a surface of the hydration liquid received by a receiver of a light beam or an audio beam transmitted from a transmitter.

4. The hydration monitor according to claim 1, wherein the volume sensor is located beneath, or at the bottom of, the container or wherein the volume sensor is located above or at the top of the container.

5. The hydration monitor according to claim 4, wherein the volume sensor includes a transmitter for transmitting a beam of ultrasonic pulses through the hydration liquid for downward reflection from an under-surface of the hydration liquid to a receiver or wherein the volume sensor is operable to derive data dependent on the current quantity of hydration liquid in the container from a change in the combined weight of the container and the hydration liquid therein, resulting from a change in the amount of hydration liquid in the container.

6-7. (canceled)

8. The hydration monitor according to claim 1, wherein the location sensor comprises a first part on or in the container and a second part at a relatively fixed location relative to the container.

9. (canceled)

10. The hydration monitor according to claim 1 comprising an orientation sensor operable to derive data dependent on the current orientation of the container.

11. (canceled)

12. The hydration monitor according to claim 1 comprising a temperature sensor operable to derive data dependent on the current temperature of the hydration liquid in the container.

13. (canceled)

14. The hydration monitor according to claim 1 comprising a combination of a container and a hub.

15. The hydration monitor according to claim 14, wherein the container and the hub are interengagable with one another.

16. (canceled)

17. The hydration monitor according to claim 1 comprising: a lid arrangable at least partially over an internal volume of the container.

18. (canceled)

19. The hydration monitor according to claim 1 comprising a hydration sensor operable to derive data dependent on the current hydration of a person in close proximity to the hydration monitor and/or comprising one or more light sources.

20. (canceled)

21. The hydration monitor according to claim 2 comprising a data transfer device operable to transmit or transfer data stored in the memory to a data processing device.

22. (canceled)

23. A system for monitoring hydration of one or more people within an area to be monitored comprising:
one or more hydration monitors, each hydration monitor being assigned to a given person within the area to be monitored;
a data processing device arranged to:
receive raw data from the one or more hydration monitors; and
process the raw data to produce one or more final reportable events;
a database arranged to store data relating to the or each final reportable event; and
a presentation engine operably connected to the database and arranged to produce a presentation utilising the data relating to one or more of the final reportable events.

24. The system for monitoring hydration according to claim 23, wherein: one or more of the hydration monitors is/are a hydration monitor comprising: a container for holding and dispensing a hydration liquid; a volume sensor operable to derive data dependent on the current quantity of the hydration liquid in the container; and a location sensor operable to derive data dependent on the current location of the container; and/or wherein the raw data received from the hydration monitor(s) are stored in an analytics engine memory.

25. (canceled)

26. The system according to claim 23, wherein: processing the raw data includes identifying one or more events of potential interest.

27-29. (canceled)

30. The system according to claim 23, wherein, in the data processing device, a processor is arranged to carry out a processing method including one or more of the following steps:
pre-processing the raw data, including validating and cleaning the raw data, to produce clean data;
analysing the clean data to identify potential events of interest;
assigning a confidence level to each potential event of interest;
checking and revising the confidence level assigned to each potential event of interest as new data are supplied to the data processing device;
if the confidence level does not pass a first confidence threshold, then the potential event of interest is considered erroneous or should be consolidated and labelled a non-event;
if the confidence level assigned to an event of potential interest passes the first confidence threshold, then the event of potential interest becomes a reportable event;
assigning a confidence level to the or each reportable event;
assessing the confidence level assigned to the or each reportable event against a second confidence threshold;
marking a reportable event as a final reportable event if the confidence level assigned to the reportable event passes the second confidence threshold;
if the confidence level does not pass a second confidence threshold, then the reportable event is considered erroneous or should be consolidated and labelled a non-event.

31. The system according to claim 23, wherein the presentation engine is arranged to augment the presentation with data from one or more other sources.

32. (canceled)

33. A method for monitoring hydration of one or more people within an area to be monitored comprising:
collecting raw data from one or more hydration monitors, each hydration monitor being assigned to a given person within the area to be monitored;
processing the raw data to produce one or more final reportable events; and
producing a presentation utilising the data relating to one or more of the final reportable events.

34. (canceled)

35. The method according to claim 33, wherein the method is performed using a system comprising:
the one or more hydration monitors;
a data processing device arranged to:
receive the raw data from the one or more hydration monitors; and
process the raw data to produce the one or more final reportable events;
a database arranged to store data relating to the or each final reportable event; and
a presentation engine operably connected to the database and arranged to produce the presentation utilising the data relating to one or more of the final reportable events.
